(19) United States
(12) Patent Application Publication
(10) Pub. No.: US 2007/0269411 A1
(43) Pub. Date: Nov. 22, 2007

(54) Porous silicon materials and devices

(76) Inventors: Wei Sun, Rochester, NY (US); Philippe M. Fauchet, Pittsford, NY (US); J. Edwards Puzas, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 11/715,819

(22) Filed: Mar. 7, 2007

Related U.S. Application Data
(60) Provisional application No. 60/781,053, filed on Mar. 10, 2006.

(51) Int. Cl. A61K 48/00; A61P 19/08

(52) U.S. Cl. 424/93.2; 424/423; 424/600; 424/93.7; 435/320.1; 435/325; 435/366; 514/2; 514/44; 623/23.76; 977/906

(57) ABSTRACT

Provided are materials and devices comprising physiologically acceptable silicon. The materials and devices can comprise a vector, including a viral vector.

This application claims the benefit of U.S. Provisional Application No. 60/781,053, filed Mar. 10, 2006, which is hereby incorporated herein by reference in its entirety.

### BACKGROUND

Orthopedic implant materials formed from porous silicon have been suggested for the fixation, fusion, reconstruction, treatment, and replacement of human and animal bones. Porous silicon, however, has not been optimized for use within human and animal subjects for orthopedic or other biomedical applications.

### SUMMARY

Provided are materials and devices comprising physiologically acceptable silicon. The silicon can have a plurality of pores. One or more pores can have a diameter of between about 50.0 nanometers (nm) and about 10.0 microns (μm). The materials and devices can comprise a vector. The vector can be a viral vector.

Other systems, methods, and aspects and advantages of the invention will be discussed with reference to the Figures and to the detailed description.

### BRIEF DESCRIPTION OF THE DRAWINGS

The invention will be described by way of example, in the detailed description, with particular reference to the accompanying Figures in which:

FIG. 1(a-c) shows SEM images of the surface morphology of three forms of PSi. FIG. 1a shows MacPSi with straight pores with openings greater than 1.0 μm. FIG. 1b shows MesPsi with branching pores with pore openings less than 100 nm. FIG. 1c shows NanPsi with spongy porous structure with pore sizes less than 20 nm.

FIG. 2(a-c) shows the adhesion, metabolic activity and biomarkers of osteoblasts on PSi (n=3). FIG. 2a shows a direct count of osteoblasts stained by propidium iodide on PSi and a control surface after incubation of 0.5, 1, 2 and 4 hours. FIG. 2b shows results of a cell viability assay of osteoblasts cultured on PSi and control after 4, 120 and 168 hours (luminescence was normalized to substrate area). FIG. 2c shows gene expression of alkaline phosphatase, osteocalcin and type I collagen in ROS osteoblasts cultured on PSi and a control surface for 7 days (data are normalized to β-actin level).

FIG. 3(a-f) shows cell and extracellular matrix on MacPSi substrates. FIG. 3a shows osteoblasts adhere and spread out after 18 hours of incubation (stained by propidium iodide dye). FIG. 3b shows cultured osteoblasts cluster to form nodules after 5 days (stained by propidium iodide dye). FIG. 3c shows immunofluoresence of type I collagen after 1 week of culture (Rhodamine fluorescence). FIG. 3d shows immunofluoresence of osteoclacin after 2 weeks of culture (Rhodamine fluorescence). FIG. 3e shows an SEM image of a matured osteoblast surrounding a fiberous mesh after 1 week of culture. FIG. 3f shows fibrils with banding characteristics of type I collagen after 1 week of culture.

FIG. 4(a-c) shows the mineralization of extracellular matrix on PSi. FIG. 4a shows an SEM image of a protein layer on MacPSi. FIG. 4b shows an SEM image of a protein layer shown in FIG. 4a. FIG. 4c shows the ratio of the major atoms detected (Si is from the substrate and Au is introduced by sputtering for the visualization).

FIG. 5 shows ALP activities of osteoblasts grown on virus coated and non-coated MacPSi (n=3 and error bars represent standard errors). Ad-BMP: osteoblasts cultured on Ad-BMP coated MacPSi at 50:1, 10:1, and 1:1; Ad-GFP: osteoblasts cultured on Ad-GFP coated MacPSi (10:1); No Ad: osteoblasts cultured on non-coated MacPSi.

FIG. 6 shows new bone formation on implants (n=5 and error bars represent standard errors). The increased bone volume is normalized to the initial volume.

FIG. 7 shows EDX of implants (insets are corresponding SEM images). a) EDX of MacPSi implanted subcutaneously; b) EDX of MacPSi implanted in tibia.

FIG. 8 shows EDX of bone adjacent to MacPSi and implanted MacPSi. a) EDX of bone marrow space (left) and cortical bone (right) that are adjacent to the implanted MacPSi (corresponding SEM image in the middle); b) SEM images of implanted MacPSi pin (left) and highly calcified region on the pin (middle) and EDX of the highly calcified region (right).

FIG. 9 shows histology of new bone formation in marrow space (the implanted pins have been removed before staining): the red is the bone matrix, the dark purple is the bone marrow, the white is the empty space, and the blue arrow points at the bone-porous silicon interfaces. a) the control without any implant; b) the Si implant; c) the MacPSi implant; d) the Ad-BMP coated implant.

FIG. 10 shows TRAP staining of bony tissue around an Ad-BMP coated MacPSi implant (the implant has been removed before staining). Arrows point at remodeling sites that are stained by TRAP.

### DETAILED DESCRIPTION

As used herein, the singular forms “a,” “an” and “the” include plural referents unless the context clearly dictates otherwise. Thus, for example, reference to a “particle” includes aspects having two or more such particles unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

Tissue engineering strategies using engineered biomaterials that support and promote tissue growth can be used for reconstructive surgeries and other biomedical applications. The goal of tissue engineering is to repair, generate, or regenerate human or animal tissue with biomaterials based medical devices.

Silicon (Si), a semiconductor material can be used in tissue engineering. Due to its wide use in the microelectronic industry, the physical and chemical properties of Si are widely known.

The disclosed materials and devices comprise physiologically acceptable silicon. The silicon can have a plurality of pores. One or more pore can have a diameter of between about 50.0 nanometers (nm) and about 10.0 microns (μm). For example, at least one pore can have a diameter of between about 500 nm and about 5 μm. In another example, at least one pore can have a diameter of between about 1.0 μm and about 2.0 μm. At least one pore can also be less than 50 nm in diameter.

The silicon can be resorbable. By “resorbable” it means that the silicon can be fully or partially absorbed by a subject's body over a period of time. The silicon can be selected from one or more of: bioactive silicon, resorbable silicon, biocompatible silicon, porous silicon, polycrystalline silicon, amorphous silicon, and bulk crystalline silicon. The porous silicon can be derivatised porous silicon. The derivatised porous silicon may comprise a Si—C or a Si—O—C covalent link. The derivatised porous silicon may comprise a carbon chain.

For the purposes of this specification a “physiologically acceptable” is a material that is biologically acceptable for specific applications including, but not limited to, the implantation of the described materials into a subject's body.

The porous silicon can further comprise one or more channel. A channel can have the same structure as a pore, but can have a larger diameter. The channel can be longer or of the same length as a pore. Channels can also comprise pores. For example, pores can be located on the inner surface of one or more channel. Thus, the silicon surface that defines a channel can be formed from silicon having pores with a diameter of between about 50.0 nanometers (nm) and about 10.0 microns (μm). Pores and channels can range from, for example, about 50 μm to at least about 100 mm in length or depth. For example, the pores and channels can be between about 0.5 mm to about 80 mm in length or depth.

The channels can have a diameter greater than about 10.0 μm. Optionally, the diameter of at least one channel can be greater than about 10.0 μm but less than about 300 μm. For example, the diameter of at least one channel can be between about 100 μm and about 300 μm.

Pores and channels can be formed in silicon using methods known in the art. For example, silicon can be micromachined and/or etched. The pores and channels can also be formed using laser energy, ultrasound or air abrasion. Thus, the described silicon device and materials can be formed using a variety of known processes. Optionally, the pores are formed by etching and the channels are formed by ultrasonic microdrilling. If the pores and channels are not circular in cross section, then the diameters referred to apply to the largest diameter of the cross sectional shape of the pore or channel. Moreover, sophisticated microfabrication techniques allow precise structures to be formed from silicon substrates. For example, etching, dicing, laser ablation, ultrasound, and air abrasion can all be used to generate structures comprising silicon.

Porous Silicon (PSi), a “derivative” of silicon, can have a structure of void pores mixed with microcrystalline and/or nanocrystalline silicon. PSi can be generated by electrochemical etching of a silicon substrate. A large range of pore configurations, including, for example, pore width, depth (up to the full thickness of the initial substrate), and porosity (20%-90% or more or less) can be obtained.

According to the International Union of Pure and Applied Chemistry (IUPAC) classification of pore size, PSi can be grouped in three classes: 1) microporous silicon with pore width no larger than 2 nm; 2) mesoporous silicon with pore width in the range of 2 nm to 50 nm; 3) macroporous silicon with pore width larger than 50 nm. PSi can be formed by an electrochemical anodization of Si in a hydrofluoric acid (HF)-based etchant. Although electroless etching technique such as stain etching can be also used for this purpose, electrochemical etching can have better control on the morphology of resulting PSi. In one example, the silicon materials and devices can be macroporous.

A series of exemplary etching conditions that can be used to obtain exemplary pores, is shown in Table 1.

TABLE 1

| | | | |
|---|---|---|---|
| pore scale | Nano- | Meso- | Macro- |
| pore size | 1-5 nm | 5-100 nm | 0.5-2 μm |
| morphology | spongy with | pores with | straight pores |
| | nano/micro | branches | and rods |
| | crystals | | |
| Si doping | 5-20 | 0.001 | 5-20 |
| level (Ω-cm) | | | |
| electrolytes | 10%-30% | 10%-30% | 4%-8% |
| | HF/ethanol | HF/ethanol | HF/DMF |
| current density | 8-25 | 8-50 | 2-10 |
| (mA/cm²) | | | |

Table 1 shows exemplary etching conditions for different types of porous silicon. HF: hydrofluoric acid; DMF: dimethylforamide. The exemplary conditions described in Table 1 can be used to prepared three types of PSi: nano-scale (<15 nm, NanPSi), meso-scale (approximately 50 nm, MesPSi) and macro-scale (approximately 1 μm, MacPSi) pores. PSi samples can be produced by electrochemical etching of silicon wafers in hydrofluoric acid (HF) based electrolytes. The various pore configurations can be achieved by changing the Si substrate, the electrolyte content, or the current density.

Boron-doped p<100> silicon wafers (550 μm thick with a resistivity of 20-30 ohm-cm) can be used for etching MacPSi and NanPSi. Silicon wafers with a resistivity of 0.008-0.012 ohm-cm can be used for MesPSi. Wafers for MacPSi can be pre-doped on the backside to form a p+ conductive layer for contact purpose. PSi can be prepared using an anodization process in a custom-designed single etching cell. An electrolyte of 4 wt. % hydrofluoric acid (HF) in dimethylformamide (DMF) can be used for the anodization of MacPSi and an electrolyte of 15 wt. % HF in ethanol can be used for MesPSi and NanPSi etching. For MacPSi, the wafer can be etched with a current density of 2 mA/cm² for about 30 minutes. MesPSi and NanPSi can be etched with a current density of 10 mA/cm² for about 10 minutes. Then the PSi samples can be cleaved into 1×1 cm² chips and rinsed with ethanol and deionized water sequentially. The chips can be oxidized by immersing them in H₂O₂ (30%) overnight to protect them from natural aging.

MacPSi and MesPSi samples can be characterized by scanning electron microscopy (SEM), while NanPSi samples can be characterized by atomic force microscopy (AFM). MacPSi can have pores with openings approximately 1 μm; MesPSi can have pores with openings around 50 nm; and NanPSi can have a spongy porous structure with pore sizes under 15 nm.

One exemplary method to form pores is to etch pores on silicon particles that are larger than the pores. Stain etching can be used for this purpose. Another exemplary method is to use smaller sized silicon particles to form a structure such that empty spaces between particles perform the function of pores. The shape, size, spacing, and array of the powders can be determined for the osteoconductivity and degradability. To further enhance the drug delivery function of the material, smaller pores can be formed on those particles.

To mold particles, chemical bonds can be introduced by chemical modifications of silicon particles. Hydrogels and other polymers can be used as glues to this purpose. Compression and other molding techniques can also be used.

Moreover, the porous silicon materials in the forms of films and powders can be utilized for biomedical applications that do not require critical mechanical support from the material. Drug delivery, soft tissue repair, cancer treatment, bone repair, and cartilage repair can all be accomplished using the described materials and methods.

The material and devices can further comprise a human or animal cell. The cell can be located in a pore. The cell, whether located in a pore or otherwise, can be a stem cell. The stem cell can be a mesenchymal stem cell or an embryonic stem cell. The materials and devices can comprise a variety of human or animal cells. Optionally, the cell is selected from the group consisting of: an osteoblast, an osteocyte, a fibroblast, a red blood cell, a white blood cell, a lymphocyte, a monocyte, a macrophage, a chondroblast, a chondrocyte, a neuroblast, and a neuronal cell. The given cell comprising the material or device can be determined by one skilled in the art based on the desired application for the material or device. For example, if an orthopedic application is desired, an osetoblast, an osteocyte, a fibroblast, a chondroblast, a chondrocyte, a mesenchymal stem cell, embryonic stem cell, or a combination thereof can be selected. Alternatively, if a neurologic application is desired, a neuroblast, neural cell, mesenchymal stem cell, embryonic stem cell, or combinations thereof can be used. Similarly, if a hematopoietic application is desired, a red blood cell, a white blood cell, a lymphocyte, a monocyte, a macrophage, an embryonic stem cell, mesenchymal stem cell, or combinations thereof can be used.

Stem cells are defined (Gilbert, (1994) DEVELOPMENTAL BIOLOGY, 4th Ed. Sinauer Associates, Inc. Sunderland, Mass., p. 354) as cells that are “capable of extensive proliferation, creating more stem cells (self-renewal) as well as more differentiated cellular progeny.” These characteristics can be referred to as stem cell capabilities. Pluripotential stem cells, adult stem cells, blastocyst-derived stem cells, gonadal ridge-derived stem cells, teratoma-derived stem cells, totipotent stem cells, multipotent stem cells, embryonic stem cells (ES), embryonic germ cells (EG), and embryonic carcinoma cells (EC) are all examples of stem cells.

Stem cells can have a variety of different properties and categories of these properties. For example in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express October 4 and Nanog (Rodda et al., J. Biol. Chem. 280, 24731-24737 (2005); Chambers et al., Cell 113, 643-655 (2003)). It is understood that some stem cells will express these at the mRNA level, and still others will also express them at the protein level, on for example, the cell surface or within the cell.

The materials and devices comprising stem cells can have any combination of any stem cell property or category or categories and properties discussed herein. For example, some stem cells can express alkaline phosphatase, not express SSEA-1, proliferate for at least 20 passages, and be capable of differentiating into any cell type. Another set of stem cells, for example, can express SSEA-1 on the cell surface, and be capable of forming endoderm, mesoderm, and ectoderm tissue and be cultured for over a year without differentiation. Another set of stem cells, for example, could be pluripotent stem cells that express SSEA-1. Another set of stem cells, for example, could be blastocyst-derived stem cells that express alkaline phosphatase.

Stem cells can be cultured using any culture means which promotes the properties of the desired type of stem cell. For example, stem cells can be cultured in the presence of basic fibroblast growth factor, leukemia inhibitory factor, membrane associated steel factor, and soluble steel factor which will produce pluripotential embryonic stem cells. See U.S. Pat. Nos. 5,690,926; 5,670,372, and 5,453,357, which are all incorporated herein by reference for material at least related to deriving and maintaining pluripotential embryonic stem cells in culture. Stem cells can also be cultured on embryonic fibroblasts and dissociated cells can be re-plated on embryonic feeder cells. See for example, U.S. Pat. Nos. 6,200,806 and 5,843,780 which are herein incorporated by reference at least for material related to deriving and maintaining stem cells.

The materials and devices can comprise a pluripotential embryonic stem cell. A pluripotential embryonic stem cell as used herein means a cell which can give rise to many differentiated cell types in an embryo or adult, including the germ cells (sperm and eggs). Pluripotent embryonic stem cells are also capable of self-renewal. Thus, these cells not only populate the germ line and give rise to a plurality of terminally differentiated cells which comprise the adult specialized organs, but also are able to regenerate themselves.

The materials and devices can comprise stem cells which are capable of self renewal and which can differentiate into cell types of the mesoderm, ectoderm, and endoderm, but which do not give rise to germ cells, sperm or egg.

The materials and devices can comprise stem cells which are capable of self renewal and which can differentiate into cell types of the mesoderm, ectoderm, and endoderm, but which do not give rise to placenta cells.

The materials and devices can comprise an adult stem cell which is any type of stem cell that is not derived from an embryo or fetus. Typically, these stem cells have a limited capacity to generate new cell types and are committed to a particular lineage, although adult stem cells capable of generating all three cell types have been described (for example, United States Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are referred to as “pluripotent hematopoietic stem cell” for its pluripotency within the hematopoietic lineage. A pluripotent adult stem cell is an adult stem cell having pluripotential capabilities (See for example, United States Patent Publication no. 20040107453, which is U.S. patent application Ser. No. 10/467,963.

The materials and devices can comprise a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

The materials and devices can comprise a gonadal ridge-derived stem cell which is a pluripotent stem cell which was derived from a cell which was obtained from, for example, a human embryo or fetus at or after the 6, 7, 8, 9, or 10 week, post ovulation, developmental stage. Alkaline phosphatase staining occurs at the 5-6 week stage. Gonadal ridge-derived stem cell can be derived from the gonadal ridge of, for example, a 6-10 week human embryo or fetus from gonadal ridge cells.

The materials and devices can comprise an embryo derived stem cell which is derived from embryos of 150 cells or more up to 6 weeks of gestation. Typically embryo derived stem cells will be derived from cells that arose from the inner cell mass cells of the blastocyst or cells which will be come gonadal ridge cells, which can arise from the inner cell mass cells, such as cells which migrate to the gonadal ridge during development.

The materials and devices are generally described by reference to “stem cells” or “pluripotent stem cells.” However, the disclosed materials and devices are not limited to use of stem cells and pluripotent stem cells. It is specifically contemplated that the disclosed methods and compositions can use or comprise any type or category of stem cell, such as adult stem cells, blastocyst-derived stem cells, gonadal ridge-derived stem cells, teratoma-derived stem cells, totipotent stem cells, and multipotent stem cells, or stem cells having any of the properties described herein. The use of any type or category of stem cell, both alone and in any combination, with or in the disclosed materials and devices is specifically contemplated and described.

Pluripotent stem cells maintained, for example, on feeder layers and with appropriate culture medium remain undifferentiated indefinitely. Removal from the feeder layer and culture in suspension leads to the formation of aggregates and other differentiated cells (Kyba, M, (2003) Meth. Enzymol. 365, 114-129). These aggregates begin to organize and develop some of the characteristics of blastocysts. These protoblastocysts are called embryoid bodies (EB). Within the EB, progressive rounds of proliferation and differentiation occur, roughly following the pattern of development. While a wide variety of tissue types can be identified in EBs, without outside direction, differentiation is disorganized and does not lead to formation of significant quantities of any one cell type (Fairchild, P J, (2003) Meth. Enzymol. 365, 169-186). Numerous strategies have been devised to direct a larger proportion of cells down any particular developmental pathway (Wassarman, P M, Keller, G M. (2003) METHODS IN ENZYMOLOGY, Differentiation of Embryonic Stem Cells, vol. 365, Elsevier Academic Press, New York, N.Y., 510p.). These have taken the form of treatment with known morphogens, alteration of the hormonal environment, culture of the cells on particular substrata, and sequential application of chemicals known to affect differentiation in vitro. All of these strategies have been successful in certain applications but in no case have they been able to generate cells that are homogenously one cell type.

In order for stem cell derived products to be applied in real applications, large quantities of identical cells can be to be generated. Ideally, this can be a general process that could be applied broadly rather than necessitating tedious experimentation for each cell type.

Other than stem cells, the materials and devices can comprise cells of the human or animal body. Cells of the human or animal body include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting melanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squarnous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), Thymus epithelial cell

One or more cell comprising the materials or devices can produce extracellular matrix. Thus, the materials and devices can further comprise extracellular matrix. The extracullular matrix can comprise calcium and phosphorous. For example, the extracellular matrix can have a calcium:phosphorous ratio of greater than 1.17. For example, the calcium phosphorous ratio can be between about 1.50 and about 1.80.

The materials and devices can also comprise a pharmacologic agent or combinations thereof. As used herein, “pharmacological agent” means a compound that can have a therapeutic effect is a subject when administered, exposed or otherwise contacted with the subject. A therapeutic effective amount refers to the quantity of active pharmacological agent sufficient to yield a desired therapeutic response or effect without undue adverse side effects such as toxicity, irritation, or allergic response. Therapeutic effect includes but is not limited to any effect on a normal physiological or pathological event, process, structure, composition, or portions, or combinations thereof of a subject. The effective therapeutic amount can vary with such factors as the particular condition being treated, the physical condition of the patient, the type of animal being treated, the duration of the treatment, the nature of concurrent therapy (if any), and the specific formulations employed and the structure of the compounds or its derivatives. The optimum effective amounts can be readily determined by one of ordinary skill in the art using routine experimentation.

As used herein, pharmacological agents can be any type of molecule or compound that can have a therapeutic effect. Thus, for example, a pharmacological agent can include but is not limited to a protein, amino acid, peptide, polypeptide, nucleic acid, or any other compound or composition, or any fragments or portions thereof, which can have a therapeutic effect in a subject. Pharmacological agents can be or be derived from exogenous pharmacological agents or endogenous pharmacological agents. Thus, the term pharmacological agent is not limited by the origin of the agent.

Exemplary pharmacological agents can be selected from the exemplary group consisting of: a growth factor, a morphogenetic protein, an antimicrobial agent, a fluoride, a vitamin D metabolite, calcitonin, raloxifene, estrogen, and a hormone. If a growth factor is used, the growth factor can selected from the exemplary group consisting of: bone morphogenetic protein 2, bone morphogenetic protein 3, bone morphogenetic protein 4, bone morphogenetic protein 5, bone morphogenetic protein 6, bone morphogenetic protein 7, bone morphogenetic protein 8, bone morphogenetic protein 9, bone morphogenetic protein 10, bone morphogenetic protein 11, bone morphogenetic protein 12, bone morphogenetic protein 13, bone morphogenetic protein 14, transforming growth factor beta, transforming growth factor beta isoform 1, transforming growth factor beta isoform 2, transforming growth factor beta isoform 3, vascular endothelial growth factor (VEGF), insulin-like growth factor I (IGF-I) insulin-like growth factor II (IGF-II), fibroblast growth factor (FGF2), platelet derived growth factor isoform PDGFaa, platelet derived growth factor isoform PDGFbb, platelet derived growth factor isoform PDGFab.

Some pharmacological agents can promote the bone growth process. BMP, VEGF, OPN, PTH, and Vitamin D, for example, can be used to coat PSi, including, MacPSi. Some pharmacologic agent can produce an anti-cancer effect in a subject having a cancer. Coating of multiple drugs can further promote bone generation or anti-cancer effects.

Using silicon powder-based biomaterials as an example, one particle can be coated with one type of pharmacological agent, and a second particle with another pharmacological agent. Biomaterials made of multiple particles can have multiple therapeutic effects.

The materials and devices can further comprise a vector. The vector can comprise at least one nucleic acid sequence encoding a pharmacologic agent. Thus, provided herein are materials and devices, comprising physiologically acceptable silicon and a vector, wherein the vector comprises at least one nucleic acid sequence encoding a pharmacological agent. The vector can be a viral vector, for example, an adenoviral vector. In some examples, the vector can encode a pharmacological agent that can promote the bone growth process or that can produce an anti-cancer effect in a subject having a cancer.

Pharmacological agents as described above can be encoded by the nucleic acid of the vector. The pharmacological agent encoded by the nucleic acid sequence of the vector can be a therapeutic protein or a therapeutic portion thereof. An exemplary pharmacological agent encoded by the nucleic acid sequence can be selected from the exemplary group consisting of: a growth factor, a morphogenetic protein, an antimicrobial agent, a fluoride, a vitamin D metabolite, calcitonin, raloxifene, estrogen, osteopontin (OPN), receptor activator for nuclear factor kB ligand (RANKL), parathyroid hormone (PTH) and a hormone. If a growth hormone is encoded, the growth factor can be selected from the exemplary group consisting of: bone morphogenetic protein 2, bone morphogenetic protein 3, bone morphogenetic protein 4, bone morphogenetic protein 5, bone morphogenetic protein 6, bone morphogenetic protein 7, bone morphogenetic protein 8, bone morphogenetic protein 9, bone morphogenetic protein 10, bone morphogenetic protein 11, bone morphogenetic protein 12, bone morphogenetic protein 13, bone morphogenetic protein 14, transforming growth factor beta, transforming growth factor beta isoform 1, transforming growth factor beta isoform 2, transforming growth factor beta isoform 3, vascular endothelial growth factor (VEGF), insulin-like growth factor I (IGF-I) insulin-like growth factor II (IGF-II), fibroblast growth factor (FGF2), platelet derived growth factor isoform PDGFaa, platelet derived growth factor isoform PDGFbb, platelet derived growth factor isoform PDGFab.

The vector of the material can contact a cell of the subject. The nucleic acid of the vector can be expressed by the contacted cell. Many vectors capable of contacting a subject cell and having a nucleic acid sequence capable of expression by the contacted cell are known in the art. The material can comprise any of these known vectors. Moreover, the material can comprise any vector known, or not known, that is capable of delivering a nucleic acid sequence to a subject. One exemplary vector that the material can comprise is a viral vector. For example, the material can comprise an adenoviral vector. In one example, the vector is Ad-BMP-2. A vector can be attached to a portion of the silicon. The silicon comprising the vector can be used in a device that is implantable within a subject.

Similar to the selection of cellular types described above, the selection of a desired pharmacological agent, vector or any combination thereof can be made by one skilled in the art based on the desired application for the materials or devices. Thus, for example, growth factors or hormones know to affect bone metabolism can be selected for orthopedic applications alone or in combination. Similarly, anti-cancer agents can be used for cancer treatment. Moreover, some pharmacological agents can be used for multiple applications. For example, antimicrobial agents can be desirable for multiple applications and can be incorporated into the materials and devices for use in many different anatomical sites within a subject.

The exact amount of cells or pharmacologic agent used may vary from subject to subject, depending on the species, age, weight and general condition of the subject, the severity of the disorder being treated, the cell or agent used, its mode of administration and the like. Thus, it is not possible to specify an exact amount for every cell or agent. However, an appropriate amount can be determined by one of ordinary skill in the art using only routine experimentation given the teachings herein.

“Treatment” or “treating” means to use the disclosed materials and/or devices in a subject with a condition, wherein the condition can be any pathologic disease or condition. The effect of the use to the subject can have the effect of but is not limited to reducing the symptoms of the condition, a reduction in the severity of the condition, or the complete ablation of the condition.

The described materials can be used to form a variety of physiologically acceptable devices. A device can be selected from the group consisting of: a pin, a nail, a screw, a plate, a staple, a tack, an anchor, a fiber, a mesh, a scaffold, a powder, and a fixation block.

Whether or not a device is selected from the preceding group of exemplary devices, the device can have a longitudinal dimension and a shorter cross sectional dimension. For example, a device having a longitudinal dimension and a shorter cross sectional dimension can be substantially cylindrical or rod-like in shape. The cross-sectional shape of the cylindrical or rod-like device can be square or rectangular. Moreover, the shorter cross sectional dimension can between about 0.25 mm and about 25.0 mm and the longitudinal dimension is between about 1.0 mm and about 80.0 mm. The desired dimensions can be selected based on the desired application. For example, in an orthopedic application, the dimensions can depend on the site where the device will be used. Thus, a larger dimensioned device can be used in a large bone in a subject such as a human as compared to a device for use in a small bone of a smaller subject such as a mouse. By non-limiting example a device used in a mouse can be approximately 0.5 to 1.0 mm in diameter by 3.0 to 5.0 mm long, a device used in a rat can be approximately 1.0 to 1.5 mm in diameter by 3.0 to 10 mm long, a device used in a rabbits can be approximately 1.5 to 2 mm in diameter by 5.0 to 15 mm long, a device used in a dogs can be approximately 2.0 to 5 mm in diameter by 15 to 35 mm long and a device used in a humans can be approximately 10 to 20 mm in diameter by 25 to 75 mm long. If the cross section along the shorter dimension is not circular, then the diameter referred to above can instead refer to the longest cross sectional length across the shorter dimension.

A disclosed device can also be irregular, substantially spherical or spheroid, or substantially cuboid in shape. If the device is irregular or substantially cuboid it can have a largest lengthwise dimension of between about 4.0 μm and about 1.0 mm. If the device is substantially spherical or spheroid, then the diameter or largest lengthwise dimension can be between about 4.0 μm and about 1.0 mm. In some examples, the largest lengthwise dimension or diameter is between about 4 μm and 100 μm. Optionally, the largest lengthwise dimension or diameter is between and about 4 μm, 40 μm and 100 μm.

The described porous silicon can be biocompatible, biodegradable, and/or osteoconductive. The large surface of the material can be modified chemically so that its surface properties can be tailored according to the application and various drugs can be immobilized on it. For example, silanization can be performed to add NH₂ groups to the surface of the PSi. Additional chemical or biochemical molecules can be attached to the NH₂ groups or to the surface. Electricity and optics can be used to enhance the performance of the described medical devices. For example, electrical field, sound or optics can be used to control the porous silicon based device to attract host cells and adjust their behavior as well as to release embedded drugs to promoted tissue regeneration. Therefore, the silicon component of the implant can be used as a transducer to control the release of the attached drugs and/or direct stimulation to the damaged bone.

Thus, the micro/nano-architecture of PSi can regulate cell behavior in vivo. The surface chemistry of PSi is flexible so that the interfacial properties between this material and living cells can be tailored by chemical modifications. PSi can support and promote primary osteoblast growth, protein matrix synthesis, and mineralization. Moreover, the osteoconductivity of PSi and other cellular responses can be controlled by altering the micro/nano architecture of porous interface. The materials can be used for both scaffolding and drug delivery functions.

Cells, including osteoblasts, can have increased adhesion and/or metabolic activity on the disclosed materials and devices as compared to other silicon or tissue engineering materials. For example, a greater number or density of cells can attach to the materials and devices than on other materials at a comparable time. The adhesion of osteoblasts or other cells to the disclosed device and materials can be quantified, for example, by direct counting of the attached cells.

Attached cells can also demonstrate increased viability. Increased viability can be demonstrated by, for example, an increased measurement of adenosine triphosphate (ATP) content in cells at a given time period as compared to other materials or a control material. Thus, cells attached to the disclosed devices and materials can have a higher ATP content at a given time than a cell attached to another silicon material or tissue engineering material, or control material at a comparable time.

Cells attached to the disclosed materials and devices can also have increased gene expression demonstrating increase viability on the disclosed devices and materials. For example, alkaline phosphatase, osteocalcin, and/or type I collagen gene expression in cells attached to the disclosed device and materials can be increased when compared to gene expression in cells attached to another silicon or tissue engineering materials or to a control at a comparable time after attachment. Know methods can be used to measure increased gene expression including, but not limited to, real-time PCR(RT-PCR).

Cells attached to the disclosed devices and materials can also produce calcified extra-cellular matrix (ECM) layers. The disclosed materials and devices can increase the amount of ECM produced by attached cells and the mineralization of the ECM. For example, after a comparable period of cellular attachment, the disclosed devices and materials can comprise a higher level of produced ECM, and the ECM produced can have a higher calcium:phosphorous ratio than, cells grown on other silicon materials, or on other tissue engineering materials or control materials. For example, the Ca to P ratio of ECM on the disclosed devices and material can be higher at a given time than the Ca:P ratio on other silicon materials demonstrating an increased formation of an apatite-like material. Thus, the ECM on the disclosed materials and devices can have an increased mineralization than ECM on other materials at a comparable time after attachment of matrix producing cells. The amount of ECM can be quantified by know methods, for example, by using immunofluoresecnce techniques. The mineralization of extra-cellular matrix on the disclosed devices and materials can also be determined by know methods. For example, fluorescent microscopy, or electron scanning electron microscopy (SEM) can be used. The atoms comprising the ECM can also be detected, by using, for example, Energy Dispersive X-ray (EDX) spectrum techniques. The atomic ratio of the major elements can be obtained by quantifying the spectra.

The described materials and devices can be used for a bone graft substitute. For example, the materials can be machined to cylinders or beams for bone grafting. The resulting graft can be inserted into the medullary cavity of the broken bone parts so that the broken bones are reconnected. The structural properties of the graft can be tailored by tuning the porous architecture.

The materials and devices can also be used as a scaffold for bone repair and regeneration. For example, the scaffold can be shaped according the geometry of a subject's broken bone. Bone forming cells (osteoblasts) or stem cells from the subjects themselves can be used as seeds to be immobilized in the PSi-based scaffold. The scaffold provides both the structural support to the damaged tissue and the vehicle to deliver the cells. The described porous architectures are designed so that the surface will support and promote the cell growth and the integration of the implant into the host tissue. Moreover, the PSi-based materials and devices are gradually degraded within the subject.

The materials and devices can provide both scaffolding and controllable drug delivery functions. For example, drugs or biomolecules that can stimulate tissue regeneration are integrated into a scaffold comprising the disclosed materials. The drugs can be released on site when the materials are degraded. The release can also be controlled by an electric field or direct current that is loaded on the device.

The materials and devices can be molded to form orthopedic implants. For example, a plurality of silicon particles are molded into a desired structure according to the structure of a damaged bone. The space between particles provides the graft with porous architecture to load cells to repair the damaged tissue. The micro porous environments can be supplied by chemical etching of the particles so that pores are formed on materials and devices. The mechanical properties of the graft can be controlled by the molding conditions.

The materials and devices can be used as a paste for spine fusion. The micro scaled silicon or PSi particles described above can be modified by chemical treatment so that they can be loaded with bone forming cells and bind to the intervertebral disks. In this manner, the dysfunctional intervertebral disks are fused by bony tissues that formed by the bone forming cells. The particles can be introduced to the intervertebral disks by spraying or they can be coated onto the both side of a biodegradable film that will serve as scaffold between intervertebral disks.

The material can also be implanted into the marrow space of bone (where blood cells are forming) and can be used to establish hematopoietic repopulation. Cells that can be used for this application can include red blood cells, lymphocytes, monocytes and macrophages.

In some examples, the materials and devices relate to orthopedic implant materials, to orthopedic implant devices comprising said materials and to methods of fabrication of said materials and devices.

The materials and devices can be used for a range of applications relating to the fixation, fusion, reconstruction, treatment, and replacement of human and animal bones. Conditions treated in this way include bone fractures, bone degeneration, and bone cavities caused by events such as trauma and infection.

Perhaps the most commonly used orthopedic implant materials are titanium and stainless steel. These materials can be used, for example, in the treatment of fractures. The fractured bone or bones being held together by screws and/or plates formed from the metal. Another material that has been used in bone fixation is self reinforced poly(glycolic acid) (SR-PGA). Screws formed from SR-PGA have been used in the treatment of cancellous bone fractures; an advantage of SR-PGA being its resorbability.

Bone replacements, such as joint replacements used in the treatment of arthrosis of the hip and knee, include orthopedic implant material such as polymethylmethacrylate which is used as a bone cement in the replacement. Bone cavities resulting from such things as trauma and tumors are typically treated by autografting. The autograft harvest, however, can result in considerable patient discomfort.

The described materials and devices allow for good integration between the material and the bone to prevent loosening of the implant. Such loosening can be caused by infection or by reaction to the presence of the implant in the subject's body. For many applications it is advantageous that the implant material should minimize the risk of such infection or adverse reaction. The risk of loosening can also be reduced by encouraging the bonding or growth of bone and supporting soft tissue to or into the implant.

The materials and devices used in the repair of a bone can be used for the duration of the repair. The use of resorbable materials that are absorbed by a patient's body over a period of time can last during this repair period. By having an implant that is absorbed, expensive and time consuming surgery removing the implant can be avoided. A beneficial substance, such as an antimicrobial agent or bone growth factors can be incorporated in the resorbable material to be released as the material corrodes.

The described materials and devices can be used in the treatment and/or repair and/or replacement of animal or human bone. The bone may require such treatment and/or repair and/or replacement as a result of damage, disease, or a genetic defect.

The term replacement is intended to include the growth of a bone or part of a bone that was not present in a subject's body. The materials and devices can be adapted for use within an animal or human. It can also be adapted for use outside an animal or human body. For example, bone repair could be performed outside a subject's body, the repaired bone or bones then being replaced in the patient by surgery. The materials and devices can be used to fix bones or bone portions together, it may form part of a scaffold to encourage bone growth across a gap between bones or to encourage regrowth of a damaged bone, and it can be used as a shield to preventingrowth of soft tissue in the space between bones or bone portions.

The use of porous and/or polycrystalline silicon promotes calcification and hence bone bonding. The semiconductor properties of porous and/or polycrystalline silicon opens the way for electrical control of the treatment, repair, or replacement process.

The disclosed materials and devices can have a structure and composition such that it is suitable for use in the treatment of one or more of the following conditions: hip fracture, arthrosis of the hip and knee, vertebral fracture, spinal fusion, long bone fracture, soft tissue repair, and osteoporosis.

The use of resorbable porous and or polycrystalline silicon can obviate the need for surgery to remove the orthopedic implant material. The porous and/or polycrystalline silicon is corroded in the body during the replacement of the bone. Porous and/or polycrystalline silicon also has a high mechanical strength, and is therefore more suitable for load bearing applications. The corrosion properties of porous silicon can be tailored to those required for a particular implant by controlling the pore size of the material. The use of resorbable silicon is advantageous since the corrosion of porous and/or polycrystalline silicon results in the formation of silicic acid, a chemical that has been shown to stimulate bone growth.

The materials and devices can comprise derivatised porous silicon. More advantageously the derivatised porous silicon comprises Si—C and/or Si—O—C bonding.

The described materials and devices can be used as an orthopedic implant device formed, at least partly, from the described materials comprising porous and/or polycrystalline silicon. The materials and devices can be used in the treatment, and/or replacement, and/or the repair of bone in an animal or human patient. The materials and devices can have a structure and composition such that it can be used for the fixation of human cortical bone fractures. The materials and devices can also have a structure and composition such that it is suitable for the treatment of one or more of: hip fracture, vertebral fracture, spinal damage, craniofacial damage, and long bone fracture.

The materials and devices can comprise a biasing means for electrically biasing at least part of the porous and/or polycrystalline silicon. The biasing means can comprise a means for generating current flow through the materials and/or device. The biasing means may comprise a battery.

The materials and device can further comprise animal and/or human bone. The materials and device can comprise autografted animal or human bone. The materials and device can comprise a scaffold that encourages bone repair or replacement. The scaffold can comprise collagen.

Advantageously the materials and device comprise a micromachined component, the structure and composition of said micromachined component being such that interaction between the materials and device and surrounding tissue and cells is enhanced relative to use of the device without the micromachined component.

Also provided are methods of treating and/or repairing and/or replacing and/or fixing and/or reconstructing bone comprising implanting the silicon materials or devices into a region of an animal or human body requiring treatment and/or replacement and/or repair and/or reconstruction and/or fixation of bone and allowing bone to grow onto at least part of the surface of the silicon. Further provided is a method of treating and/or repairing and/or replacing and/or fixing and/or reconstructing bone comprising implanting silicon materials or devices into a region of an animal or human body to assist with treatment and/or replacement and/or repair and/or reconstruction and/or fixation of bone and allowing the silicon to resorb.

For applications such as the treatment of human or animal bones, the growth of bone into the structure can be desirable. Pores and channels can be used into which bone can grow. Channels formed in the interior of the silicon structure or in the surface of the structure may be interconnected to facilitate growth of the bone into the structure and/or bonding of the bone to the structure.

Silicon can be porosified by standard techniques. For example, silicon can porosified by anodisation in aqueous or ethanolic HF, or it can be porosified by stain etching. The silicon materials and devices can comprise bioactive porous amorphous silicon and one or more of: titanium and stainless steel. Preferably the porous amorphous silicon forms at least part of an orthopedic implant material. The use of orthopedic implants comprising porous amorphous silicon may be of value for the treatment or reconstruction of bone since it is a relatively straight forward to coat metals and other materials with amorphous silicon. Porosification of silicon formed at the surface of the implant may confer bioactivity to the implant, allowing to bond with bone or other living tissue.

The materials and devices are intended to interact with the biological environment into which they are introduced. Such biomaterials can be bio-inert, bioactive or resorbable, depending on their interaction with the living tissue of the human or animal body.

The disclosed devices and materials can also comprise a plurality of physiologically acceptable silicon particles. Each particle can have a plurality of pores and one or more pore can have a diameter of between about 50 nm and about 10.0 μm. For example, at least one pore can have a diameter of between about 500 nm and about 5 μm. In another example, at least one pore can have a diameter of between about 1.0 μm and about 2.0 μm. At least one pore can also be less than 50 nm in diameter.

Each porous silicon particle can further comprise one or more channel. A channel can have the same structure as a pore, but has a larger diameter. The channel can be longer or of the same length as a pore. Channels can also comprise pores. For example, pores can be located on the inner surface of one or more channel. Thus, the silicon surface that defines a channel can be formed from silicon having pores with a diameter of between about 50.0 nanometers (nm) and about 10.0 microns (μm). Pores and channels can be formed in silicon using methods know in the art. For example, the methods described above can be used. If the pores and channels are not circular in cross section, then the diameters referred to apply to the largest diameter of the cross sectional shape of the pore or channel.

The at least one channel can have a diameter greater than about 10.0 μm. Optionally, the diameter of at least one channel can be greater than about 10.0 μm but less than about 300 μm. For example, the diameter of at least one channel can be between about 100 μm and about 300 μm. Pores and channels can range from, for example, about 50 μm to at least about 100 mm in length or depth. For example, the pores and channels can be between about 0.5 mm to about 80 mm in length or depth.

Each particle can be irregular in shape. Alternatively, each particle can be substantially spherical or spheroid or substantially cuboid. Moreover, the devices and materials can comprise combinations of irregular, substantially spherical or spheroid, and substantially cuboid particles. If the particles are irregular or substantially cuboid they can have a largest lengthwise dimension of between about 4.0 μm and about 1.0 mm. If the particles are substantially spherical or spheroid, then the diameter or largest lengthwise dimension can be between about 4.0 μm and about 1.0 mm.

The plurality of particles can be used to form at least a portion of a physiologically acceptable device. For example, a plurality of particles can be molded to form a physiologically acceptable or medical device or a portion thereof. To mold a plurality of particles together to form a device or a portion thereof, the materials and devices can further comprise a bonding material. A bonding material can be resorbable meaning that it can be absorbed by a subject's body over a period of time. Thus, a plurality of the silicon particles can be bound to each other by a resorbable bonding material. The bonding material can be a polymer. For example, the polymer can be an epoxy. The bonding material can also be a biological material. The biological material can be selected from the group consisting of: collagen matrix, poly lactic acid and a fibrin clot. Both natural and synthetic polymers used for bone and tissue engineering can be used as the bonding material. For example, collagen, fibrin, chitosan, starch, hyaluronic acid, poly(hydroxybutyrate), poly(α-hydroxy acids), poly(ξ-caprolactone), poly(propylene fumarates), poly(BPA iminocarbonates), poly(phosphazenes) and poly(anhydrides) can be used.

A physiologically acceptable or medical device comprising a plurality of particles can be selected from the group consisting of: a pin, a nail, a screw, a plate, a staple, a tack, an anchor, a fiber, a mesh, a scaffold, a powder, and a fixation block.

Whether or not a device is selected from the preceding group of exemplary devices, the device can have a longitudinal dimension and a shorter cross sectional dimension. For example, a device having a longitudinal dimension and a shorter cross sectional dimension can be substantially cylindrical or rod-like in shape. The cross-sectional shape of the cylindrical or rod-like device can be square or rectangular. Moreover, the shorter cross sectional dimension can between about 0.25 mm and about 25.0 mm and the longitudinal dimension is between about 1.0 mm and about 80.0 mm. The desired dimensions can be selected based on the desired application. For example, in an orthopedic application, the dimensions can depend on the site where the device will be used. Thus, a larger dimensioned device can be used in a large bone in a subject such as a human as compared to a device for use in a small bone of a smaller subject such as a mouse. By non-limiting example a device used in a mouse can be approximately 0.5 to 1.0 mm in diameter by 3.0 to 5.0 mm long, a device used in a rat can be approximately 1.0 to 1.5 mm in diameter by 3.0 to 10 mm long, a device used in a rabbits can be approximately 1.5 to 2 mm in diameter by 5.0 to 15 mm long, a device used in a dogs can be approximately 2.0 to 5 mm in diameter by 15 to 35 mm long and a device used in a humans can be approximately 10 to 20 mm in diameter by 25 to 75 mm long. If the cross section along the shorter dimension is not circular, then the diameter referred to above can instead refer to the longest cross sectional length across the shorter dimension.

The materials and devices comprising plurality of particles can further comprise a human or animal cell. The cell can be attached to at least one particle. Optionally, the cell can be located in a pore that is located on at least one particle. Similar to the description above, the materials and devices comprising a plurality of particles can comprise a variety of human or animal cells. The cell can be stem cell, including a mesenchymal stem cell or an embryonic stem cell. Optionally, the cell is selected from the group consisting of: an osteoblast, an osteocyte, a fibroblast, a red blood cell, a white blood cell, a lymphocyte, a monocyte, a macrophage, a chondroblast, a chondrocyte, a neuroblast, and a neuronal cell. The given cell comprising the material or device can be determined by one skilled in the art based on the desired application for the material or device. For example, if an orthopedic application is desired, an osetoblast, an osteocyte, a fibroblast, a chondroblast, a chondrocyte, a mesenchymal stem cell, embryonic stem cell, or a combination thereof can be selected. Alternatively, if a neurologic application is desired, a neuroblast, neural cell, mesenchymal stem cell, embryonic stem cell, or combinations thereof can be used. Similarly, if a hematopoietic application is desired, a red blood cell, a white blood cell, a lymphocyte, a monocyte, a macrophage, an embryonic stem cell, mesenchymal stem cell, or combinations thereof can be used. Other stem cells and cells as described above can be used.

One or more cell comprising the materials or devices can produce extracellular matrix. Thus, the materials and devices can further comprise extracellular matrix. The extracellular matrix can have a calcium phosphorous ratio of greater than 1.17. For example, the calcium phosphorous ratio can be between about 1.50 and about 1.80.

The materials and devices comprising a plurality of particles can also comprise a pharmacologic agent or combinations thereof. Exemplary pharmacological agents include those described above and can be selected from the exemplary group consisting of: a growth factor, a morphogenetic protein, an antimicrobial agent, a fluoride, a vitamin D metabolite, calcitonin, raloxifene, estrogen, and a hormone. If a growth factor is used, the growth factor can selected from the group consisting of: bone morphogenetic protein 2, bone morphogenetic protein 3, bone morphogenetic protein 4, bone morphogenetic protein 5, bone morphogenetic protein 6, bone morphogenetic protein 7, bone morphogenetic protein 8, bone morphogenetic protein 9, bone morphogenetic protein 10, bone morphogenetic protein 11, bone morphogenetic protein 12, bone morphogenetic protein 13, bone morphogenetic protein 14, transforming growth factor beta, transforming growth factor beta isoform 1, transforming growth factor beta isoform 2, transforming growth factor beta isoform 3, vascular endothelial growth factor (VEGF), insulin-like growth factor I (IGF-I) insulin-like growth factor II (IGF-II), fibroblast growth factor (FGF2), platelet derived growth factor isoform PDGFaa, platelet derived growth factor isoform PDGFbb, platelet derived growth factor isoform PDGFab.

Similar to the selection of cellular types described above, the selection of a desired pharmacological agent, vector or any combination thereof can be made by one skilled in the art based on the desired application for the materials or devices. Thus, for example, growth factors or hormones know to affect bone metabolism can be selected for orthopedic applications. Moreover, some pharmacological agents can be used for multiple applications. For example, antimicrobial agents can be desirable for multiple applications and can be incorporated into the materials and devices for use in many different anatomical sites within a subject.

A device can also comprise a plurality of physiologically acceptable silicon particles, wherein the plurality of the particles are positioned in relation to each other to form at least a portion of the medical device. The medical device having one or more pore with a diameter of between about 50 nm and about 10.0 μm. The medical device can further comprise channels, cells and pharmacological agents as described above. The cells can be located in the pores formed between the particles. Other pore sizes can be used, including all pore sizes described herein.

The devices comprising a plurality of particles can be used in the same biomedical applications that are described above for devices not comprising a plurality of particles. Such devices can be used with a pharmacological agent and/or vector as described herein.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Nano to Micro Scale Porous Silicon Architecture

The osteoconductivity of PSi was evaluated using nano-scale (less than 15 nm, NanPSi), meso-scale (30-50 nm, MesPSi) and macro-scale (1-2 μm, MacPSi) pores in vitro. The PSi samples were produced by electrochemical etching of p-type silicon wafers in hydrofluoric acid (HF) based electrolytes. The various pore configurations were achieved by changing the Si substrate, the electrolyte content or the current density.

PSi preparation: Boron-doped p<100> silicon wafers (550 μm thick with a resistivity of 20-30 ohm-cm) were used for etching MacPSi and NanPSi. Silicon wafers with a resistivity of 0.008-0.012 ohm-cm were used for MesPSi. All PSi were prepared using an anodization process in single etching cell as described in Sun et al., A three dimensional porous silicon p-n diode for betavoltaics and photovoltaics. Adv. Mater. 17, 1230-1233 (2005).

Porous silicon was prepared using an anodization process in a single etching cell. A tungsten mesh was used as the cathode while the anode was an aluminum sheet pressed against the back side of a silicon wafer.

An electrolyte of 4 wt. % hydrofluoric acid (HF) in dimethylformamide (DMF) was used for the anodization of MacPSi and an electrolyte of 15 wt. % HF in ethanol was made for MesPSi and NanPSi etching. For MacPSi, the wafer was etched with a current density of 2 mA/cm² for 30 minutes. MesPSi and NanPSi were etched with a current density of 10 mA/cm² for 10 minutes. Then the PSi samples were cleaved into 1×1 cm² square chips and rinsed with ethanol and deionized water sequentially. The chips were oxidized by immersing them in H2O2 (30%) overnight.

Cell culture: Primary osteoblasts isolated from 2-4-day-old rats were cultured in DMEM containing low glucoses (Invitrogen, Carlsbad, Calif.) supplemented with 5% bovine serum (Hyclone Laboratories, Logan, Utah), 1% penicillin/streptomycin, 1% ascorbic acid and 1% betaglycerophosphate. The pH of the culture media was adjusted to 7.4. The ROS 17.2.8 osteosarcoma cell line was maintained with the same media except that no ascorbic acid and beta-glycerophosphate were added. All cells were allowed to proliferate in a standard incubator at 37° C. to reach confluence. Upon confluence, cells were released from the flask with trypsin-EDTA. 6×10⁴ cells in 1 ml media were seeded onto each PSi chip sitting in a standard 24-well plate. The media were refreshed every two days during the culture period.

Cell imaging: After culturing, the substrates with cells were rinsed with phosphate buffered saline (PBS) and then fixed with methanol for 7 minutes. The samples were immersed in 5 μg/ml propidium iodide (PI) dye (Sigma) for 10 minutes. For calcium labeling, PBS rinsing was performed after PI staining and then the samples were incubated with 2 μM calcein (Invitrogen, Carlsbad, Calif.) for 10 minutes. The samples were rinsed with PBS after all staining. Then fluorescence-labeled samples were excited at ˜510 nm with a fluorescent microscope for cell counting, or excited at ˜480 nm for the visualization of calcified protein matrix. Images of three randomly chosen 1×1 mm² areas were taken at 100× magnification on each sample. The number of cells was counted in each of the three images and averaged for final analysis. The sample preparation for SEM was adapted from the method described by Karp et al., Bone formation on two-dimensional poly(DL-lactide-co-glycolide) (PLGA) films and three-dimensional PLGA tissue engineering scaffolds in vitro, J. Biomed. Mater. Res. 64A, 388-396 (1997), except that the final dehydration was done by immersing the samples in hexamethyldisilazane (Polysciences, Inc., Warrington, Pa.) for 12 minutes. After drying under air, the samples were sputter-coated with a thin layer of gold for imaging purpose.

Viability assay: The CellTiter-Glo® Luminescent Cell Viability Assay (Promega, Madison, Wis.) was used for determining cell viability on PSi and control substrates. After culturing for predetermined times, the media inside the wells was removed and the chips were moved to new culture plates. The chips were then rinsed with PBS. 500 μl DMEM and 50 μl CellTiter-Glo® Reagent (Promega, Madison, Wis.) was sequentially added into each well containing a chip. The contents were then gently mixed on an orbital shaker for 2 minutes and stabilized at room temperature for 10 minutes. 200 μl of the mixture in each well was transferred to an opaque-walled 96 well plate for luminescence detection. The luminescence was read by a VICTOR2® 1420 multiable counter (Perkin Elmer Life Science, Wellesley, Mass.) with an integration time of 1 second per well.

Immunofluorescence: After culturing, the substrates with cells were transferred to a new culture plate and rinsed with PBS. The samples were immersed in 3% Hydrogen Peroxide for 10 minutes and rinsed again with PBS. Non-specific binding sites were blocked in 1:2 goat serum for 20 minutes. After aspirating the serum, samples were incubated with primary antibodies at 4° C. overnight. For osteocalcin (OC) imaging, 1:100 dilution of goat anti-rat OC antibody (Biomedical Technologies Inc., Stoughton, Mass.) was used. For type I collagen (Col1) imaging, 1:40 dilution of rabbit anti-rat Col1 antibody (Chemicon International, Temecula, Calif.) was used. Following 3 rinses with PBS, the samples were incubated with 1:50 dilution of Rhodamine conjugated donkey anti-goat secondary antibody (Rockland Inc., Gilbertsville, Pa.) for 30 minutes. The fluorescence labels were excited at 515-560 nm.

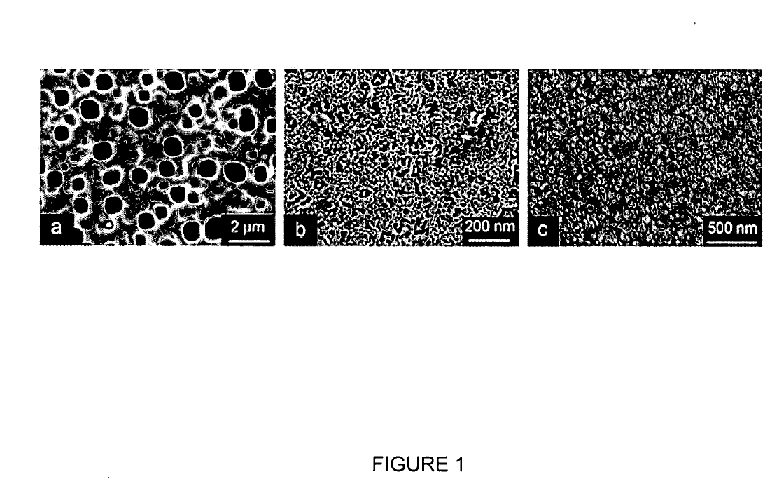

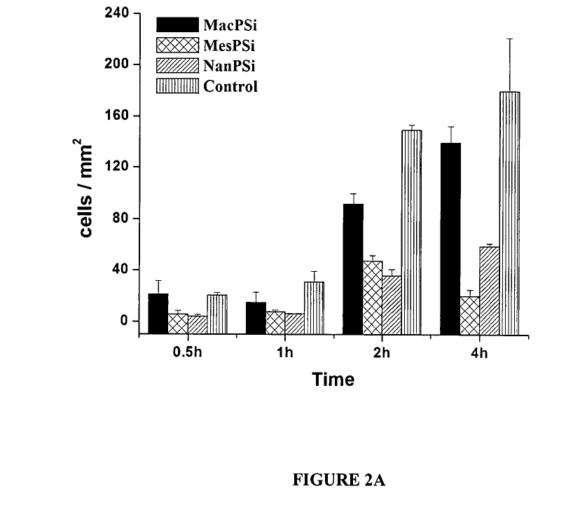

As shown in FIG. 1, MacPSi had straight pores with openings above 1 μm; MesPSi had straight but branching pores with pore openings under 100 nm; and NanPSi had a spongy porous structure with pore sizes under 10 nm. To protect PSi from gradual oxidation and degradation in air, a chemical oxidation in hydrogen peroxide was carried out after etching to form a thin oxide layer on the surface. Primary rat calvaria cells (osteoblasts) or rat osteosarcoma cells (ROS 17.2.8) were seeded onto PSi substrates for 1 hour to 5 weeks and the substrates and cells were assayed both qualitatively and quantitatively. Standard cell culture in 24-well polystyrene culture plates was used as a control.

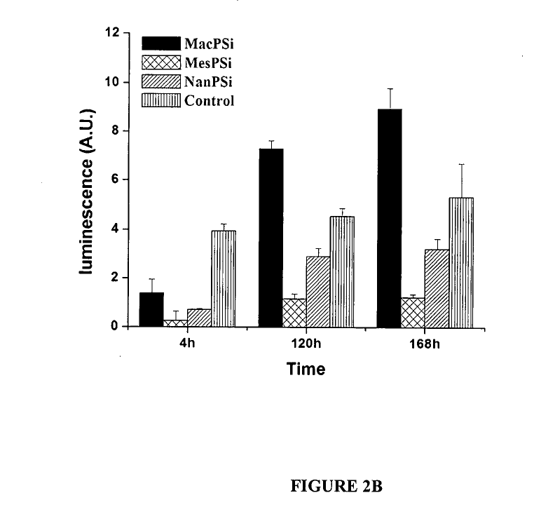

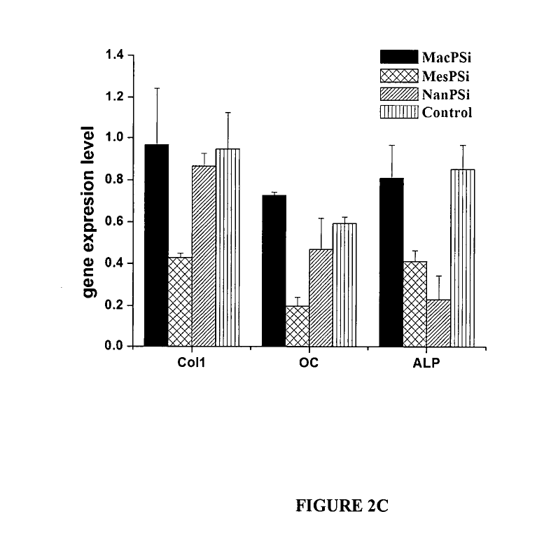

The adhesion of osteoblasts to PSi surfaces was quantified by direct counting of the attached cells. The viability of the attached cells was determined by an adenosine triphosphate (ATP)-based cell viability assay. In adhesion studies (0.5 hour to 4 hours), PSi chips bound slightly fewer osteoblasts than the tissue culture plate (FIG. 2a). Among those porous samples, MacPSi anchored the most cells, and MesPSi exhibited the lowest cell affinity. The viability assay measuring ATP content in cells was conducted 4 hours after the cells had been cultured on samples (FIG. 2b). Osteoblasts had the highest viability on MacPSi among the three forms of PSi.

At 5 and 7 days of culturing, the metabolic activity of osteoblasts was examined with the cell viability assay. As seen in FIG. 2b, lower viabilities were detected on MesPSi and NanPSi than the control. This could be attributed to the fact that fewer cells were attached to these samples initially. Higher viability was found on MacPSi than on control tissue plates at days 5 and 7, demonstrating that osteoblasts grow on MacPSi have a higher metabolic activity than those grown on the other surfaces.

To verify whether the biological functions of the osteoblasts grown on PSi were affected by the substrate, real-time PCR(RT-PCR) was employed to quantify three characteristic biomarkers of bone formation1: alkaline phosphatase (AP), osteocalcin (OC) and type I collagen (Col1).

The ROS 17.2.8 osteoblast cell line was used. After culturing these cells on PSi for 7 days, the three genes were detected on all three types of PSi (FIG. 2c). MacPSi maintained the transcription of all these biomarkers at a high level, comparable to the control surface. The osteoblasts on NanPSi exhibited a low AP transcription, but the OC and Col1 transcriptional levels were conserved. All three RNAs were found on MesPSi substrates at a moderate level compared to the control. These results show that the surface geometry of the substrates influences cell behavior.

Considering attachment, viability and gene expression MacPSi provided osteoblasts with the most favorable microenvironment to foster bone formation.

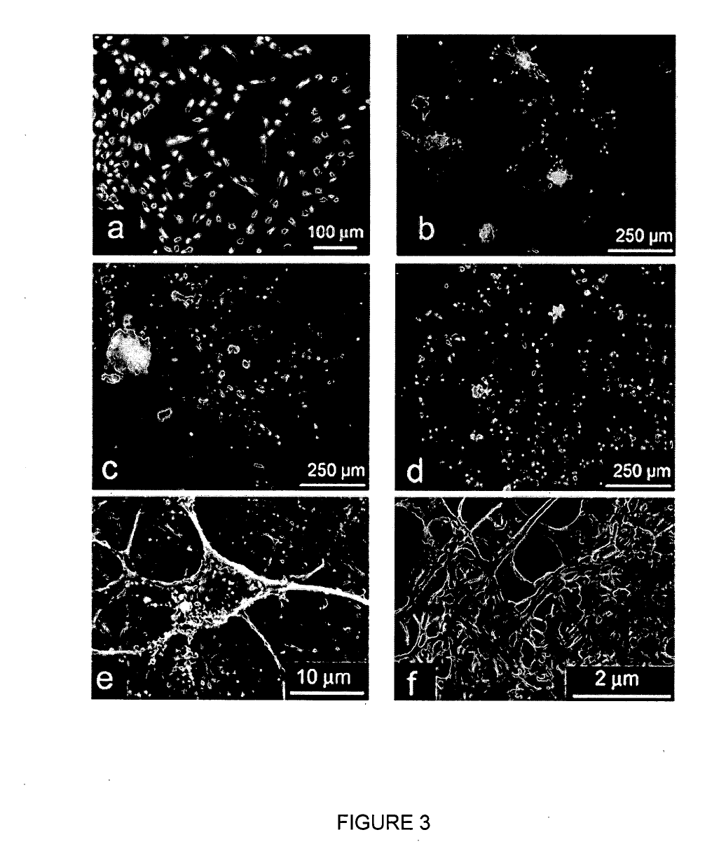

The morphology of the cultured cells on PSi was characterized by fluorescence microscopy, scanning electron microscopy (SEM), and fluorescence immunohistology. After cells adhered to substrates, they tended to spread out on the MacPSi and NanPSi surfaces (FIG. 3a), but remained more separated and rounded on the MesPSi substrates. Within 3-5 days of culture, these adhered cells migrated, proliferated and clustered to form mineralizing nodules (FIG. 3b), a feature common in the process of bone formation. Upon maturation, the osteoblasts secreted an extracellular matrix (ECM) that could support further mineralization. Type I collagen, which constitutes approximately 95% of this protein matrix in bones, was detected on all PSi samples by immunofluorescence after 1 week of culture (FIG. 3c). Osteocalcin, a major noncollagenous bone matrix protein and late marker of osteoblast maturation, was also present on all the samples after 2 weeks of culture (FIG. 3d). High resolution SEM images demonstrated the presence of a fibrous mesh around cultured osteoblasts with the banding characteristics of type I collagen (FIGS. 3e & 3f). These observations confirmed that PSi supports the growth and functionalization of osteoblasts. A semi-quantitative investigation on the mineralization of cultured osteoblasts on PSi samples further supported this finding.

After 7 days of culture, calcified ECM layers were detected on MacPSi but not on the other two types of PSi. After 2 weeks, calcified layers were found on all PSi substrates.

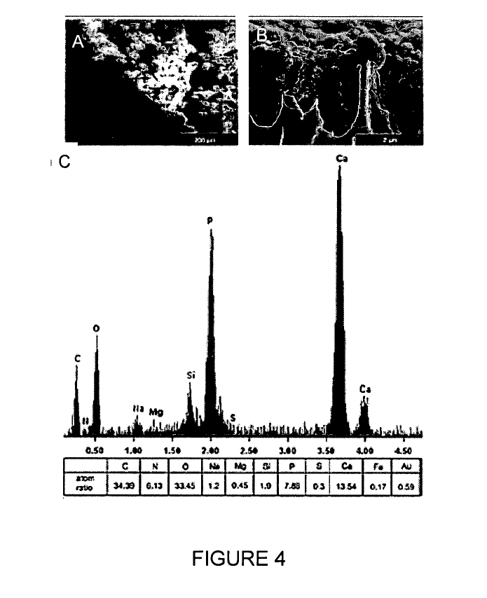

Using a dual fluorescence labeling method with propidium iodide (PI) and calcein, the cells and the mineralized matrix were simultaneously visualized. Ca-rich protein matrix with green fluorescence (stained by calcein) and osteoblasts with red fluorescence (stained by propidium iodide dye) at the mineralization front of ECM laid down by osteoblasts was demonstrated on MesPSi for 4 weeks. FIG. 4a is a SEM image of an ECM layer deposited by the osteoblasts on MacPSi, and FIG. 4b shows a cross-section of the wafer with penetration of the mineralized matrix into the pores. The corresponding Energy Dispersive X-ray (EDX) spectrum of this layer is shown in FIG. 4c. The atomic ratio of the major elements was obtained by quantifying the spectra.

The Ca to P ratio in the matrix on MacPSi was 1.72, showing the formation of an apatite-like material.

This ratio is in the range (1.65-1.77) found in human bone minerals. The Ca content was smaller with NanPSi and the smallest with MesPSi. The finding is consistent with a low OC transcriptional level in the cells. The osteoblasts cultured on MacPSi seem to differentiate and mature faster than on the other substrates.

MacPSi promoted osteoblast growth better than the other form of PSi as demonstrated by enhanced osteoblast viability (FIG. 2b) and mineralization (described above) and the maintained the expression of the biomarkers of bone formation (FIG. 2c).

The micrometer pore and the abundant flat silicon surface present around the pores on MacPSi anchor the cells firmly while providing them with enough space to spread. This topography activates a cascade of intracellular signaling pathways and thus guides the cells to proliferate and fulfill their function efficiently. In contrast, nano-scale pores on NanPSi, though they may mimic protein binding sites, may not anchor the cells firmly and provide the same mechanical signals to regulate cell behavior. The dense submicrometer pores and the very limited flat surface of MesPSi appear to hinder the spread of the bound cells and inhibit further growth. Thus, by tuning the local geometry of implant material, the mineralization and integration of the implant into a host can be controlled.

It was demonstrated that PSi displays promising osteoconductivity. Different architectures of PSi induced different cellular responses of osteoblasts in terms of adhesion, metabolic activity, protein synthesis and mineralization. MacPSi performed better than MesPSi and NanPSi in supporting osteoblast growth and sustaining their function. Considering its higher rate of mineralization, its potential biodegradability, and its potential drug delivery function, MacPSi is a compelling biomaterial for bone tissue engineering.

As described in the following examples, among porous samples, MacPSi anchors significantly more cells than the other two types after two hours of culture, while MesPSi exhibits the lowest cell affinity. Higher viability is found on MacPSi than on control tissue plates at days 5 and 7, demonstrating that more osteoblasts survived on MacPSi and/or cells have higher metabolic activities at these time points than those grown on the other surfaces. In either scenario, MacPSi is not toxic to osteoblasts and allows their growth on it.

The atomic ratio of the major elements was obtained by quantifying the spectra. The Ca to P ratio in the matrix on MacPSi was 1.72, suggesting the formation of an apatite-like material. This ratio is in the range ([1.65, 1.77]) found in human bone minerals. The Ca content was lower with NanPSi and the lowest with MesPSi. The finding is consistent with a lower OC transcriptional level in the cells. The osteoblasts cultured on MacPSi differentiate and mature faster than on the other substrates

### Example 2

### The Osteoinductivity of Porous Silicon Coated with Recombinant Adenoviral Vectors

Adenovirus-Based Gene Delivery for Bone Regeneration

Adenovirus (Ad) is a family of medium-sized (60-90 nm), nonenveloped viruses containing double-stranded DNA. It represents the largest nonenveloped virus and can accept up to 7 kb of foreign DNA. Because of the ease of production and high transduction efficiency, Ad vectors are widely used in gene transfer. Viral genes can be modified by inserting the sequence of the target gene. After virion infect host cells, host cells express viral proteins as well as the protein that the inserted gene encodes.

To minimize cytotoxicity, replication-defective Ad vectors can be engineered by deleting multiple viral genes. The virus can infect a broad array of cell types. The resulting expression can be transient.

Integrating osteoinductivity with PSi can be done by coating it with osteoinductive molecules. Because of its large internal surface area, a small volume of PSi can accommodate a large amount of such biomolecules. Ad-mediated gene therapy was used to convert infected cells to “BMP generators.”

An Ad-BMP as an osteoinductive agent was used. By coating PSi with Ad-BMP, hybrid biomaterial was achieved with both osteoconductivity and osteoinductivity. Physical absorption was employed as the coating or loading method of Ad-BMP to PSi.

### Materials and Methods

Preparation of Recombinant Adenoviral Vectors

The recombinant adenoviral vectors were prepared using ViraPower™ Adenoviral Expression System (Invitrogen, Carlsbad, Calif.). The GFP gene was inserted as a marker to test the transduction of prepared adenoviral vectors in the infection test. BMP-2 gene was used as functional gene to promote osteogenetic activity of osteoblasts grown on MacPSi.

Adenoviral stocks were aliquotted in glycerol and stored at −80° C. Prior to use, the adenoviral stocks were thawed at room temperature. The virion were counted using a DU® 640 Spectrophotometer (Beckman Coulter, Fullerton, Calif.) after lysis in SDS solution (PBS: 10% SDS: virus=98:1:1 by volume). In this method, DNA amount was directly assayed by its absorbance at wavelength of 260 nm (OD₂₆₀), and viral particles are calculated as 1 DNA unit=1011 particles/μl.

Coating Adenovirus on Porous Silicon

1×1 cm² MacPSi chips were placed in 24-well culture plates. 500 μl Ad solution diluted 10⁵-10⁷ was pipetted onto each chip or control surface. The plates stayed at room temperature under a sterilized hood for a half hour and the viral particles bound to the substrate. Then, the plates were frozen at −80° C. for at least 24 hours.

For cell culturing, the frozen samples were thawed at room temperature and then rinsed with standard osteoblast culture medium. Afterward, 10⁵ primary rat osteoblasts were cultured onto each MacPSi chips or control surface for further biochemical assays. Cells were allowed to grow on MacPSi for 1-2 days before further tests.

Adenovirus Infection Assay

Ad infection was assayed using GFP fluorescence. A MacPSi chip seated in a well of a 24-well plate was coated with 10⁷ virion. After freezing, thawing, and rinsing, the MacPSi chip was moved to a new well, and the original well was kept for further use. Primary osteoblasts were seeded into both the original well and a new well with MacPSi. Standard osteoblast culture medium was used. After 24 hours of culture, cells in both wells were rinsed with PBS, fixed with methanol, and stained with PI dye. The central region of the original well, MacPSi, and the edge regions in both wells were examined with fluorescence microscopy. All cells were stained with PI dye, which was visualized at ˜510 nm. GFP fluorescence was excited at ˜480 nm.

Digital pictures were taken at 10× magnification in each test region at both excitation wavelengths. The pictures capturing PI fluorescence and GFP fluorescence at the same site were merged using Photoshop® (Adobe, San Jose, Calif.) software for demonstration purpose. In each picture, infected cells and total cells were counted manually, and the percentage of infected cells were calculated for comparison.

Enzyme-Linked ImmunoSorbent Assay (ELISA)

A Quantikine® BMP-2 Immunoassay (R&D systems, Minneapolis, Minn.) was used to quantify the BMP-2 release from infected osteoblasts. After osteoblasts were cultured on Ad-BMP coated MacPSi for 1 to 2 days, 100 μl medium was collected from each sample and transferred to the antibody-coated microplate for ELISA. The assay was conducted following to the guideline of Quantikine® BMP-2 Immunoassay. The kidney cell line 293A cells cultured on standard plates were used as a positive control. MacPSi coated with Ad-GFP was used as a negative control. In the other negative control group, MacPSi coated Ad-BMP was cultured with medium but no cells.

Alkaline Phosphatase Activity Assay

ALP activity is an established indicator for osteoblastic activity. The assay to measure ALP activity is also well documented. After 2 days of culture, MacPSi samples were moved to a new plate. The cells were lysed by adding 200 μl mammalian protein extract into each well. After shaking the well on a shaker for 20 minutes, two 50 μl aliquots of lysate from each well were transferred to two new plates.

One of the lysates was used to determine the ALP activity by incubation with 1 ml/well 0.5 mg/ml p-nitrophenol in a standard 2-amino-2-methyl-1,3-propandiol buffer for 30 minutes. The reaction was stopped by adding 0.5 ml 0.3 M Na₃PO₄, and the optical density was measured at OD₄₀₅ with a spectrophotometer.

The cellular protein content was determined by the BCA protein assay (Pierce Chemical Co., Rockford, Ill.) according to its instructions. After samples reacted with the working reagent at room temperature for 30 minutes, the OD₅₉₅ was measured. Protein quantity was calculated against a standard curve made from bovine serum albumin. The unit of ALP activity was defined as the amount of enzyme that released 1 μmol p-nitrophenol per mg protein.

Ad-BMP was coated on MacPSi at the virus-to-cell ratios of 50:1, 10:1, and 1:1. The amount of cells initially seeded on each MacPSi chip was used to manipulate the ratios. Two control groups were used for comparison. In one group, Ad-GFP (10:1) coated MacPSi was used. In the other one, MacPSi was treated with the same procedure except that no virus has been added in glycerol.

Statistics

Each group of samples contained three individual samples. The results were labeled as mean±standard error of measurement. Data obtained at each time point was compared using t-test or one-way ANOVA. Significance set at 95%.

### Experimental Results

Adenovirus Transduction on Porous Silicon

After 24 hours of culture, green fluorescence was detected in three of four test regions. In the central region of the original well, only 6% of the attached cells displayed green fluorescence; at edge of the original well, 11% of the attached cells displayed green fluorescence; on the MacPSi chip, 20% of attached cells were green. Little green fluorescence was detected at the edge of the well containing MacPSi.

Ad vectors were coated in the original well. The central region of the surface of the well was covered by the MacPSi chip during coating process. So, only a few, if any, virion can be immobilized in this region. But, the edge portion of this well has an equal chance to anchor virion as the MacPSi chip does. In the new well, the edge portion should not have any virus before cell culture.

The results demonstrate that MacPSi can attach Ad during the coating process and the coated Ad maintain their infectivity. The observation that the percentage of infected cells for MacPSi was higher than that for the edge of the original well indicated that MacPSi anchors more Ad or Ad on MacPSi has higher infection efficiency. The low infection rate in the edge portion of the new well showed that few virion particles escape from MacPSi.

Release of BMP from Adenovirus Coated Porous Silicon

To quantify the transduction by Ad-BMP, ELISA was employed after osteoblasts were cultured on Ad-BMP coated MacPSi for 1 and 2 days. BMP-2 was detected on MacPSi coated with Ad-BMP at both time points. Very low BMP-2 signal was found on MacPSi coated with Ad-GFP, and likely represents background noise. MacPSi coated with Ad-BMP and cultured with medium but no cells, only noise-level signal was detected.

Osteoblasts cultured on Ad-BMP coated MacPSi were infected after culturing for a day. BMP-2 was expressed by those infected osteoblasts and released to medium. The expression continues at least for another day post infection. Ad-GFP also infected osteoblasts, but can not lead to increased expression of BMP-2. Ad-BMP itself can not generate BMP-2 either.

Alkaline Phosphatase Activity on Coated Porous Silicon

To gauge the overall osteoinductivity of Ad-BMP coated MacPSi, ALP activity of osteoblasts cultured on those samples was quantified. Ad-BMP to cell (initial seeding) ratios of 50:1, 10:1, and 1:1 were tested for comparison. Ad-GFP (10:1) and no virus coating were used as controls.

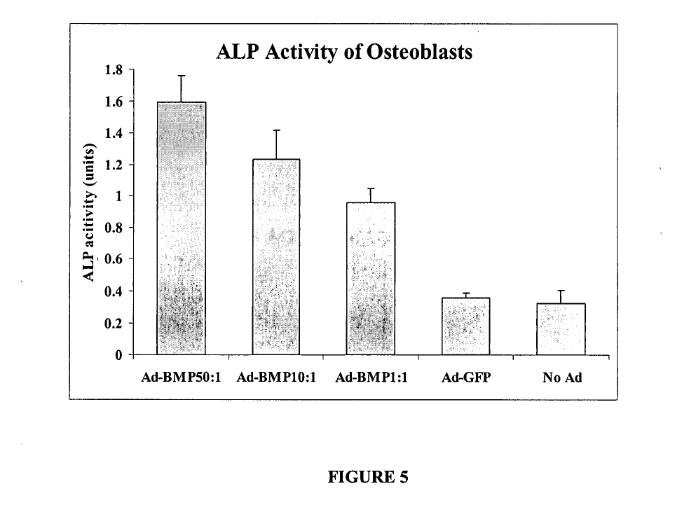

After 2 days of culture, ALP activity of osteoblasts grown on the Ad-BMP coated MacPSi was significantly higher than that of osteoblasts in the other two groups, shown in FIG. 5. Meanwhile, ALP activity of osteoblasts on Ad-GFP coated MacPSi was similar to that of osteoblasts in control group, in which no virus were coated on MacPSi. The 3-fold increase of ALP activity observed in the osteoblasts grown on Ad-BMP coated MacPSi indicates the hybrid material has osteoinductivity in vitro.

Higher dose of Ad-BMP coating also led to higher ALP activity. The results demonstrate the osteoinductivity of the hybrid material can be further tuned by controlling the initial coating dose.

### Example 3

### In Vivo Study of Bone Growth on Porous Silicon

MacPSi was used as the substrate for bone growth because of its osteoconductivity in vitro. MacPSi coated with Ad-BMP was also used to foster bone formation. Bare silicon was used as a control to study the effect of porous surface on bone growth.

### Materials and Methods

Animal Model

Two-month old mice (SV129) were used. Prior to operation, the mouse was anesthetized with 60 mg/kg ketamine and 4 mg/kg xylazine IP to provide approximately 20-30 minutes of deep anesthesia while the surgery was performed. A hole with a diameter of approximately 0.7 mm was pierced into the tibia with a needle (B-D® 22G11/2) around 5 mm below the knee. At this time a graft (3-5 mm long) that was rinsed with PBS was inserted through the intramedullary space with two ends outside of bone. Etched MacPSi was cleaved into 0.55×0.6×5 mm pins. The pins were sterilized with 70% ethanol. For Ad-BMP coating, 50 μl containing ˜10⁷ viral vectors in a 10% sorbitol-PBS solution was pipetted onto the PSi pins. The coated pin were then frozen and stored at −80° C. until transplantation.

Two groups of controls were used. In one of them, mice were treated in the same way but no implants were inserted; in the other control group, MacPSi are implanted subcutaneously.

Healing of MacPSi, Ad-BMP coated MacPSi, and bare silicon grafts were assessed at 0, 4, 6, and 8 weeks. For each treatment group, 5 mice were analyzed by micro-CT to assess the new bone formation. After sacrifice of the mice, the treated tibiae were removed and used for histology and SEM. To investigate the bone-PSi interaction, EDX technique was employed to examine the elemental composition on both implants and the bony tissue adjacent to the implants. The measurements were taken 2 weeks after surgery.

MicroCT

MicroCT was used to obtain 3D images of the treated tibiae. It allowed scanning of live mice at different times. A vivaCT 40 scanner (SCANCO Medical AG, Basserdorf, Switzerland) was used to image the tibia of the mice. High resolution (17.5 um) with x-ray settings of 55 kVp and 145 uA, an integration time of 300 ms and a cone beam reconstruction algorithm were used to scan the mice. A 3.6 mm (approximately 205 slices) region was scanned for each sample. Before scanning, the mice were anesthetized with isoflurane gas. During scanning, the mice were placed in a plastic tube with a 35.8 mm diameter and exposed to a continuous flow of isoflurane gas.

The scanned 3D images were processed with Amira® software (Amira 3.1, Mercury Computer Systems, Chelmsford, Mass.). A threshold of signal density was set at 10000 to filter the signals from soft tissues. The images were trimmed first to leave only the portions surrounding the implants. Then the images of the same sample scanned at different times were aligned using the implanted pin as the registration. The aligned images were further cropped with a confine box. The axial length (length on the z direction) was fixed at 2.2 mm. The lengths on the x and y directions were adjusted to cover the sample. Thus, the same region of the sample was chosen for comparison.

The volumes of the chosen region of images obtained at different times were calculated. In all non-control samples, at time t the total volume included the volume of inserted pin and the volume of the bone surrounding the implant, as the following equation shows:
Vtotal=Vpin-t+Vbone-t

At time 0 (right after surgery) no new bone has formed. At time 4, 6, and 8 (weeks) new bone has formed. So Vtotal changes because of the increase of Vbone-t. Then, the new bone formation can be determined by:

$$\begin{matrix}Δ Vbone = Vbone \text{-} t - Vbone \text{-} 0 \\ = ( Vpin \text{-} t + Vbone \text{-} t ) - ( Vpin \text{-} 0 + Vbone \text{-} 0 ) \\ = Vt - V 0\end{matrix}$$

Because the initial total volume and pin length were different for each sample, the new bone formation was normalized to the initial total volume for quantitative analysis. So,
New Bone Formation=ΔVbone/V0=Vt/V0−1

Histology

Standard histology was used to investigate the bone formation on implants. 8 weeks after surgery, the mice were sacrificed. Proximal tibiae with implants (or control) were dissected, fixed in 10% neutral formaldehyde, and then decalcified in 14% EDTA. After removing implants, the tibiae were embedded in paraffin. Histological sections were sliced and stained with orange-G for bone matrix and tartrate-resistant acid phosphatase (TRAP) stain for osteoclasts. Images were obtained with a microscope (Olympus, Center Valley, Pa.) and SPOT camera (Diagnostic Instruments, Sterling Heights, Mich.).

3D imaging of New Bone Formation on Implants

MicroCT images clearly show new bone formation on all three types of implants. In the control group, the hole on the tibia was filled by new bone after 4 weeks. After 8 weeks, the original area of hole became undistinguishable. No new bone formed in the marrow space. In the implant groups, after 4 weeks, the holes were sealed by new bones, which bound to implanted Si or MacPSi. Cortical bones surrounding the implant thickened, and clear evidence of remodeling was seen in these samples. Coronal sections of the 3D images reveal the new bone formation on implants in bone marrow space. No bone formation was detected on MacPSi implanted subcutaneously.

Quantification of New Bone Formation

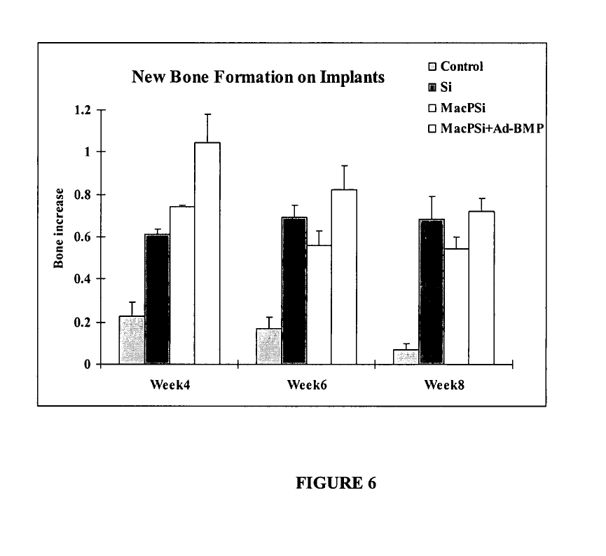

To quantify the new bone formation on implants, the increased bone volumes were calculated using the data obtained from the MicroCT scans. The result was demonstrated as the ratio of increased bone volume to the original volume of the tested region, as shown in FIG. 6.

The increased bone volumes in the implant groups were significantly higher than those in the control group. This was mainly due to the bone growth on the implant in bone marrow space. Four weeks after surgery, the Ad-BMP coated MacPSi induced the highest level of bone growth, approximately doubling the volume. At this time, bone growth on MacPSi was more than that on Si. At week 6 and 8, increased bone volume on Ad-BMP coated MacPSi and MacPSi declined, confirming that remodeling is taking place during the period. The trend was also found in the control group. As remodeling was also observed in MicroCT images of samples in the Si implant, the bone volumes have not decreased during week 4 to week 8.

Elemental Analysis of Bone Formation on MacPSi

The porous structure was only a small portion of the implant (approximately 1/25 in thickness). Thus, EDX technique was used to examine the elemental composition on both implants and the bony tissue adjacent to the implants. In this manner, the mineralization on the implant and degradation of implant was evaluated.

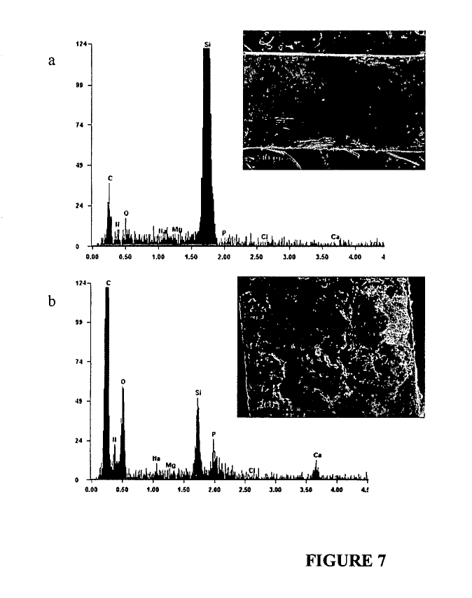

As FIG. 7 shows, more protein deposition was founded on the MacPS implanted in bone than the control implanted subcutaneously. More importantly, both Ca and P were present on the MacPSi implanted in the tibia but not on the MacPSi implanted subcutaneously. This result indicates that the tissue on the implant in tibia is calcified bone matrix.

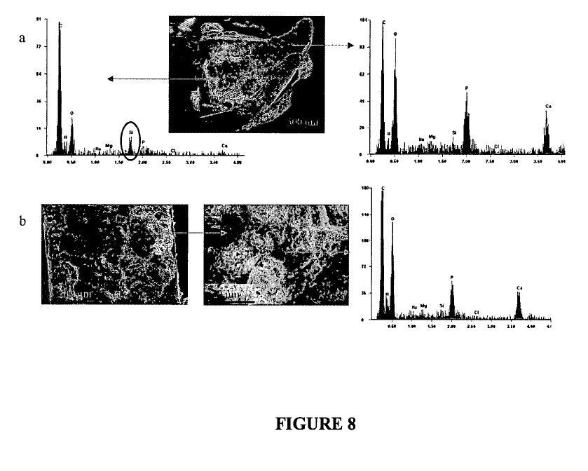

Highly calcified regions were detected on MacPSi implanted in mouse tibia, as shown in FIG. 8. In the region, the Ca to P ratio was approximately 1.4, while the ratio was 1.2 in the cortical bone adjacent to the implant. Si was also detected in the marrow space that was close to the implanted MacPSi, implying the degradation of the PSi layer. Although calcified regions were also found on the Si pins implanted in tibia, no Si was detected in the bone marrow space that was close to the implants.

Histological Analysis of Bone Formation on MacPSi

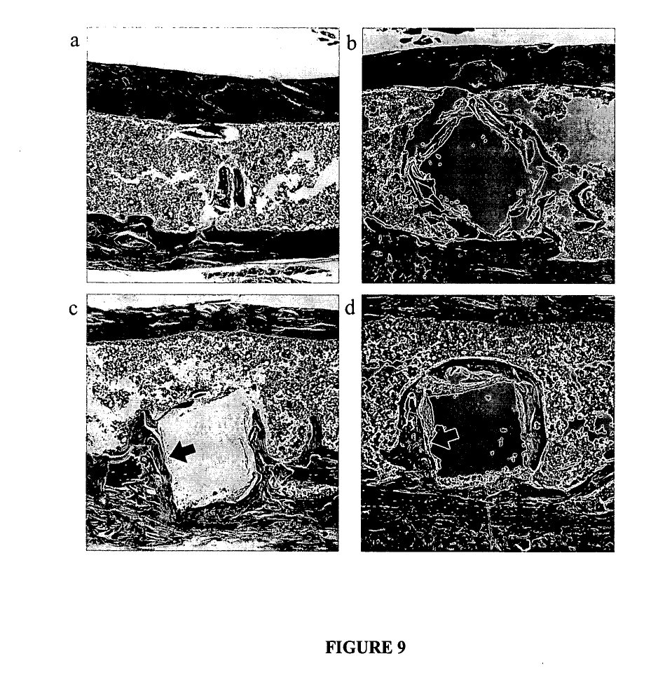

The histological analysis conducted 8 weeks after implantation revealed the detailed morphological information on the new bone formation, shown in FIG. 9. In the control group, no new bone was found in the marrow space except some bony fragments introduced by the piercing in the surgery (FIG. 9A). New bone formed on all three types of implants, following varied patterns. On the surfaces of Si implants bone formed layers that loosely connected to enclose the pin (FIG. 9B). On MacPSi implants new bone formation was prevalent on both the porous surface and the surface neighboring the cortical bone, but less significant on the other two silicon surfaces (FIG. 9C). This observation indicates that the macroporous surface bound to new bone firmly. On Ad-BMP coated MacPSi implants a tight bony coating that wraps the part of pin inside marrow space was found (FIG. 9D). Such a tightened enclosure was attributed to the osteoinduction mediated by the Ad-BMP immobilized on all the surfaces of the implants.

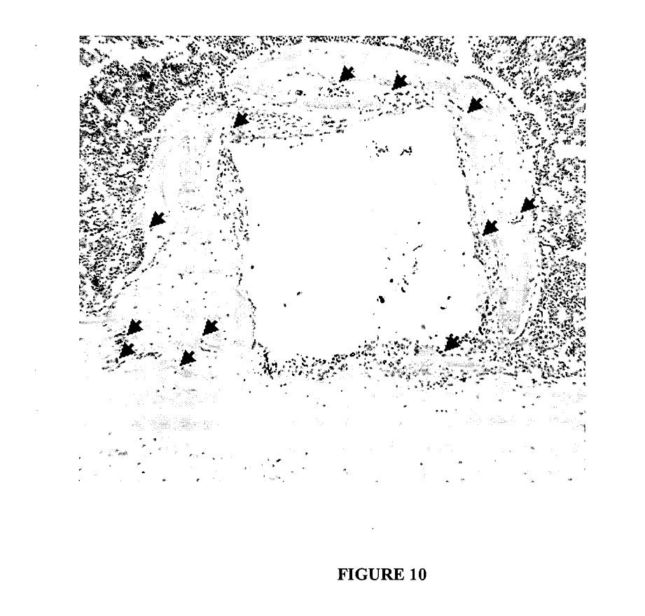

TRAP staining further demonstrated osteoclastic activity that marks bone remodeling. As FIG. 10 shows, multiple remodeling sites were founded in the new bone formed around Ad-BMP coated MacPSi. Remodeling was also detected in the bone formed around MacPSi and Si implants.

At week 4 more bone forms on Ad-BMP coated MacPSi than on Si and MacPSi, indicating the hybrid material has induced osteoinduction in vivo. The osteoinductivity of the material was also illustrated by the histology at week 8: a tight bone wrap is formed to enclose the Ad-BMP coated MacPSi in the marrow space.

The finding that a higher amount of bone tissues are grown on MacPSi than Si 4 weeks after implantation demonstrates the MacPSi surface has higher bone binding affinity. The histological analyses at week 8 further illustrate that more bone was formed on the porous surface than other surfaces.

The foregoing detailed description has been given for understanding exemplary implementations of the invention only and no unnecessary limitations should be understood there from as modifications will be obvious to those skilled in the art without departing from the scope of the appended claims and their equivalents.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Throughout this application, various publications are referenced. The disclosures of these publications in their entireties are hereby incorporated by reference into this application in order to more fully describe the state of the art to which this invention pertains.

### REFERENCES

1. Goldberg, V. M. & Caplan, A. I. (EDs) Orthopedic tissue engineering. (Marcel Dekker, Inc., NY, 2004)
2. Burg, K. J. L., Porter, S. & Kellam, J. F. Biomaterial developments for bone tissue engineering. Biomaterials 21, 2347-2359 (2000)
3. Salgado, A. J., Coutinho, O. P. & Reis, R. L. Bone tissue engineering: state of the art and future trends. Macromol. Biosci. 4, 743-765 (2004)
4. Moore, W. R., Graves, S. E. & Bain, G. I. Synthetic bone graft substitutes. ANZ J. Surg. 71, 354-361 (2001)
5. Bayliss, S. C., Heald, R., Fletcher, D. I. & Buckberry, L. D. The culture of mammalian cells on nanostructured silicon. Adv. Mater. 11, 318-321 (1999)
6. Chin, V, Collins, B. E., Sailor, M. J., & Bhatia, S. N. Compatibility of primary hepatocytes with oxidized nanoporous silicon. Adv. Mater. 13, 1877-1880 (2001)
7. Canham, L. T. et al. Derivatized mesoporous silicon with dramatically improved stability in simulated human blood plasma. Adv. Mater. 11, 1505-1507 (1999)
8. Canham, L. T. et al. Bioactive polycrystalline silicon. Adv. Mater. 8, 580-582 (1996)
9. Stewart, M. P. & Buriak, J. M. Chemical and biological applications of porous silicon Technology. Adv. Mater. 12, 859-869 (2000)
10. Letant, S. E., Hart, B. R., Buuren, A., & Terminello, L. J. Functionalized silicon membranes for selective bio-organism capture. Nature Mater. 2, 391-395 (2003)
11. Ouyang, H., Christophersen, M., Viard, R., Miller B. L. & Fauchet, P. M. Macroporous silicon microcavities for macromolecular detection. Adv. Funct. Mater. 15, 1851-1859 (2005)
12. Chan, S., Horner, S. R., Fauchet, P. M. & Miller, B. L. Identification of Gram Negative Bacteria Using Nanoscale Silicon Microcavities. J. Am. Chem. Soc. 123, 11797-11798 (2001)
13. Heiduschka, P. & Thanos, S. Implantable bioelectronic interfaces for lost nerve functions. Prog. Neurobiol. 55, 433-461 (1998)
14. Canham, L. T. Bioactive silicon structure fabrication through nanoetching techniques. Adv. Mater. 7, 1033-1037 (1995)
15. Fromherz, P. & Stett, A. Silicon-Neuron Junction: Capacitive stimulation of an individual neuron on a silicon chip. Phys. Rev. Lett. 75, 1670-1673 (1995)
16. Voskerician, G. et al. Biocompatibility and biofouling of MEMS drug delivery devices. Biomaterials, 24, 1959-1967 (2004)
17. Lai, W., Garinob, J., & Ducheynet, P. Silicon excretion from bioactive glass implanted in rabbit bone. Biomaterials, 23, 213-217 (2003)
18. Canham, L. T. (ED) Properties ofporous silicon. (IEE/INSEPC, London, 1997)
19. Buriak, J. M. Organometallic chemistry on silicon and germanium surfaces. Chem. Rev. 102, 1272-1308 (2002)
20. Hing, K. A. Bone Repair in the twenty-first century: biology, chemistry or engineering. Phil. Trans. R. Soc. Lond. A 362, 2821-2850 (2004)
21. Sun, W., Kherani N. P., Hirschman, K. D., Gadeken, L. L. & Fauchet, P. M. A three dimensional porous silicon p-n diode for betavoltaics and photovoltaics. Adv. Mater. 17, 1230-1233 (2005)
22. Karp, J. M., Shoichet, M. S. & Davies, J. E. Bone formation on two-dimensional poly(DL-lactide-co-glycolide) (PLGA) films and three-dimensional PLGA tissue engineering scaffolds in vitro, J. Biomed. Mater. Res. 64A, 388-396 (1997)

What is claimed is:

1. A material, comprising physiologically acceptable silicon having a plurality of pores, one or more pore having a diameter of between about 50 nanometers (nm) and about 10.0 microns (μm).

2. The material of claim 1, wherein at least one pore has a diameter of between about 500 nm and about 5 μm.

3. The material of claim 1, wherein at least one pore has a diameter of between about 1.0 μm and about 2.0 μm.

4. The material of claim 1, further comprising a human or animal cell.

5. The material of claim 4, wherein the cell is located in a pore.

6. The material of claim 4, wherein the cell is a stem cell.

7. The material of claim 4, wherein the material further comprises extracellular matrix.

8. The material of claim 7, wherein the extracellular matrix has a calcium phosphorous ratio of greater than 1.17.

9. The material of claim 1, further comprising a pharmacologic agent.

10. The material of claim 9, wherein the pharmacologic agent produces an anti-cancer or osteoinductive effect in a subject.

11. The material of claim 1, further comprising a vector, wherein the vector comprises at least one nucleic acid sequence encoding a pharmacologic agent.

12. The material of claim 11, wherein the pharmacological agent is a therapeutic protein or a therapeutic portion thereof.

13. The material of claim 11, wherein the pharmacological agent produces an osteoinductive effect in a subject.

14. The material of claim 11, wherein the vector is a viral vector.

15. The material of claim 12, wherein the therapeutic protein or therapeutic portion thereof produces an anti-cancer effect in a subject having a cancer.

16. The material of claim 1, further comprising a plurality of physiologically acceptable silicon particles each particle having a plurality of pores, one or more pore having a diameter of between about 50 nm and about 10.0 μm.

17. The material of claim 16, wherein at least one pore has a diameter of between about 1.0 μm and about 2.0 μm.

18. The material of claim 16, further comprising a bonding agent.

19. The material of claim 18, wherein the material can be molded into a medical device.

20. The material of claim 19, wherein the medical device is selected from the group consisting of: a pin, a nail, a screw, a plate, a staple, a tack, an anchor, a fiber, a mesh, a scaffold, a powder, and a fixation block.

21. A device, comprising physiologically acceptable silicon having a plurality of pores, one or more pore having a diameter of between about 50 nm and about 10.0 μm, wherein the device is implantable within a subject.

22. The device of claim 21, wherein at least one pore has a diameter of between about 500 nm and about 5.0 μm.

23. The device of claim 21, wherein at least one pore has a diameter of between about 1.0 μm and about 2.0 μm.

24. The device of claim 21, wherein the device is selected from the group consisting of: a pin, a nail, a screw, a plate, a staple, a tack, an anchor, a fiber, a mesh, a scaffold, a powder, and a fixation block.

25. The device of claim 21, further comprising a human or animal cell.

26. The device of claim 25, wherein the cell is attached to at least one pore.

27. The device of claim 25, wherein the cell is a stem cell.

28. The device of claim 25, wherein the material further comprises extracellular matrix.

29. The device of claim 28, wherein the extracellular matrix has a calcium phosphorous ratio of greater than 1.17.

30. The device of claim 21, further comprising a pharmacologic agent.

31. The material of claim 30, wherein the pharmacologic agent produces an anti-cancer or osteoinductive effect in a subject.

32. The device of claim 21, further comprising a vector, wherein the vector comprises at least one nucleic acid sequence encoding a pharmacologic agent.

33. The device of claim 32, wherein the pharmacological agent is a therapeutic protein or a therapeutic portion thereof.

34. The device of claim 32, wherein the pharmacological agent produces an osteoinductive effect in a subject.

35. The device of claim 32, wherein the vector is a viral vector.

36. The device of claim 33, wherein the therapeutic protein or therapeutic portion thereof produces an anti-cancer effect in a subject having a cancer.

37. The device of claim 15, further comprising a plurality of physiologically acceptable silicon particles, each particle having a plurality of pores, one or more pore having a diameter of between about 50 nm and about 10.0 μm, wherein the plurality of the particles are positioned in relation to each other to form at least a portion of the medical device.

38. The device of claim 37, wherein at least one pore has a diameter of between about 1.0 μm and about 2.0 μm.

39. A device, comprising a plurality of physiologically acceptable silicon particles, wherein the plurality of the particles are positioned in relation to each other to form at least a portion of the device, the device having one or more pore with a diameter of between about 50 nm and about 10.0 μm.

40. The device of claim 39, wherein at least one pore has a diameter of between about 1.0 μm and about 2.0 μm.

41. A material, comprising physiologically acceptable silicon and a vector, wherein the vector comprises at least one nucleic acid sequence encoding a pharmacological agent.

42. The material of claim 41, wherein the pharmacological agent is a therapeutic protein or a therapeutic portion thereof.

43. The material of claim 41, wherein the pharmacological agent produces an osteoinductive effect in a subject.

44. The material of claim 41, wherein the vector is a viral vector.

45. The material of claim 44, wherein the viral vector is an adenoviral vector.

46. The material of claim 42, wherein the therapeutic protein or therapeutic portion thereof produces an anti-cancer effect in a subject having a cancer.

47. The material of claim 41, wherein the material comprises Ad-BMP-2.

48. The material of claim 41, wherein the vector is attached to a portion of the silicon.

49. A device, comprising the material of claim 41, wherein the device is implantable within a subject.

50. A material, comprising a plurality of physiologically acceptable silicon particles each particle comprising physiologically acceptable silicon and a vector, wherein the vector comprises at least one nucleic acid sequence encoding a pharmacological agent.
